(19) United States
(12) Patent Application Publication
(10) Pub. No.: US 2008/0312669 A1
(43) Pub. Date: Dec. 18, 2008

(54) SURGICAL INSTRUMENT AND METHOD

(76) Inventors: Luc de Vries, Diepenheimthe (NL); Wouter van Furth, Hilversum (NL); Eric Windeler, Toronto (CA)

Correspondence Address: HOWREY LLP - DC, C/O IP DOCKETING DEPARTMENT, 2941 FAIRVIEW PARK DR, SUITE 200, FALLS CHURCH, VA, 22042-2924, US

(21) Appl. No.: 12/122,506

(22) Filed: May 16, 2008

Related U.S. Application Data
(63) Continuation-in-part of application No. 10/599,503, filed on Sep. 9, 2008.
Application No. 10/599,503 is a continuation-in-part of application No. 11/025,727, filed on Dec. 29, 2004.
(60) Provisional application No. 60/938,549, filed on May 17, 2007.

(30) Foreign Application Priority Data
Mar. 31, 2004 (NL) 1025852

(51) Int. Cl. A61B 17/28

(52) U.S. Cl. 606/148; 606/207; 128/898

(57) ABSTRACT

The invention relates to a surgical instrument, e.g. forceps, and method to be used for suturing tissue, the surgical instrument comprising a first arm and a second arm that are spring-connected at a proximal end, while at a distal end the first arm and the second arm can be moved towards each other, and wherein at least the first arm and/or the second arm can be provided with a bullet that is suitable for receiving and affixing a surgical needle, wherein the bullet is designed for being positioned at the distal end, at an inside and/or lower side of the end of an arm.

### CROSS REFERENCE TO RELATED APPLICATIONS

The present application claims the benefit of U.S. Provisional Application No. 60/938,549, filed May 17, 2007, and is a continuation-in-part of co-pending U.S. application Ser. No. 10/599,503, filed Sep. 29, 2006 which is the National Stage of International Application No. PCT/US2005/003476, filed Mar. 29, 2005, which is a continuation-in-part of U.S. application Ser. No. 11/025,727, filed Dec. 29, 2004 and which claims the benefit of NL 1025852, filed Mar. 31, 2004, each of which is incorporated herein by reference its entirety.

### FIELD OF THE INVENTION

The invention relates to a surgical instrument to be used for suturing tissue while reducing the possibility of needle perforation accidents.

### BACKGROUND

Standard suturing instruments and techniques present significant risks to both patient and surgeon by way of possible glove perforation accidents in which a suture needle penetrates the surgeon's glove. Such perforation accidents may allow pathogenic organisms such as, but not limited to, the hepatitis virus B, the hepatitis virus C and the human immunodeficiency virus (HIV) to be transmitted from the patient to the practitioner.

Conversely, a perforation accident may cause a break in the sterile barrier between practitioner and patient, which increases the risk of the patient's wound becoming infected.

Suturing forceps for minimally invasive (also called endoscopic, robotic, or laparoscopic) surgery, include a thin, elongated barrel with moveable arms (or jaws), for grasping and releasing tissue at one end and a control handle for manipulating the jaws (i.e. to open and close the jaws) at the other end. The user grasps the tissue with endoscopic forceps by closing the jaws around the tissue, then, using a needle holder, inserts the needle into and through the tissue requiring suturing. The user then opens the jaws of the needle holder to release the needle. Next, the same instrument or a different instrument (or instruments) is used to grasp the needle and pull it through the tissue.

During the suturing process, the surgeon must release and re-grasp the needle many times. The difficulty of suturing in endoscopic surgery is increased significantly compared to conventional surgery as all the needle-handling is done remotely by jaws. The user must be careful not to drop the needle, since the needle may be difficult to locate in the transmitted image and is difficult to pick up once the needle is found. A dropped needle must be found and picked up as it poses a serious health risk to the patient.

One approach to help avoid this problem involves the use of surgical forceps of the kind described in U.S. Patent Application US 2003/0045833 A1. The surgical forceps described in that application has near the distal end at the outside of an arm of the forceps a flexible material that can be used for manipulating a surgical needle during suturing in order to attempt to prevent needle perforation accidents.

Among the drawbacks of these and other surgical forceps is that when suturing using a surgical needle and a suture attached thereto, the tissue may sustain damage. This is a particular liability where delicate tissue is concerned through which it is difficult to pass the surgical needle without causing tissue damage. The point of the surgical needle initially pushes the tissue forward to subsequently lance it, which causes damage to the tissue. Moreover, in the surgical forceps described in the above application, the placement of the flexible material requires that the instrument be pushed further into the wound thereby increasing the likelihood of damaging the tissue and/or previously tied sutures.

Thus, there remains a need for suturing forceps, which minimize the risk of glove perforation accidents and tissue damage.

### SUMMARY OF THE INVENTION

The present invention is directed to a surgical instrument (suturing forceps) useful in conventional surgery, endoscopic and robotic surgery. The instrument comprises a first arm and a second arm that are connected at a proximal end (optionally spring-connected) so as to bias the arms in an open configuration and which define a space between them which can be reduced or increased. At a distal end, the arms can be moved towards each other thereby reducing the space between the arms. The instrument further comprises a surgical needle-receiving and affixing portion herein referred to as a “bullet” at the distal end of at least the first arm and/or the second arm, at an inside and/or lower side of the distal end of an arm. Preferably the bullet forms part of the tissue-gripping surface of the forceps. The bullet may be removable from the instrument.

The invention is further directed to the forceps described above comprising an arm manipulating means by which one arm of the instrument, preferably the arm lacking the bullet may move away from the surgical field during the suturing procedure.

Specifically, the manipulated arm is removed from the path defined by the movement of the surgical needle during suturing, which is itself defined by the curvature of the needle. Such manipulation of an arm avoids contact with the tissue being sutured thereby preventing tissue damage and facilitating the suturing process.

This may be accomplished using a variety of means including, for example, using a hinge mechanism fixed at the proximal or distal end of at least one arm of the instrument, as described in more detail below. The bullet can be placed at the distal end of the first arm and/or at the distal end of the hinge mechanism. The hinge is designed to provide a means for drawing the distal end of one of the arms away from the surgical field (preferably the arm opposite the bullet) as the distance between the distal ends of the arms increases. Conversely, as the distance between the distal ends of the arms decreases, the distal end of the arm acted upon by the hinge mechanism extends so that the arms become of similar length as the distal ends of the arms make contact with each other.

The invention is further directed to a method for suturing tissue using the instrument of the present invention comprising the steps of 1) securing and supporting a first area of tissue to be sutured with the distal ends of the instrument, 2) securing a surgical needle and a suture material attached thereto with a needle holding tool, 3) piercing the first area of tissue to be sutured with the needle using the bullet to support the tissue, 4) passing the needle through the tissue into or onto the bullet, 5) releasing the needle from the needle holding tool, 6) releasing the first area of tissue secured by the distal ends of the instrument, 7) guiding the affixed needle with the instrument following the curvature of the needle, 8) removing the needle from the bullet with the needle holding tool, and 9) repeating steps 1-8 on a second area of tissue to be sutured to the first area of the lesion whereupon the suture passing through the first and second areas of tissue is tied in a knot. Alternatively, prior to the bullet receiving and affixing the surgical needle, the surgical needle in one movement may pierce the first and second areas of tissue to be sutured. The suture is knotted by a force applied by the needle holding tool pulling on the end of the suture and a second pulling force applied by the instrument to which the surgical needle is affixed. It is to be understood that the method is also for suturing tissue using endoscopic forceps of the present invention in which case the method may also be performed robotically. The method of suturing described herein is much faster and easier than conventional suturing methods.

According to an important aspect of the present invention, the bullet is provided as or on a disposable item that can be affixed to a standard medical forceps. In one important embodiment, the bullet is provided on a disposable forceps-like device that can itself be affixed to a standard medical forceps or other suitable manipulative device.

Of importance, the disposable portion is preferably embodied as a single use component. Reuse may be prevented by ensuring that a connection, formed between the bullet or its holder and the forceps or manipulator, is broken once the connection is opened. The bullet may then be disposed of together with the suture needle on finishing the suture procedure.

According to a yet further aspect of the invention, the bullet is provided as part of a surgical system comprising all those items required for performing suturing. The surgical system may comprise a bullet as herein described, with or without a holder, together with a surgical needle and suture. An appropriate dispensing package may be provided for conveniently dispensing a number of needles and sutures to the surgeon during the suturing procedure. In particular the suture may be of the atraumatic type in which an eyeless needle and suture are combined. Such a system may facilitate suitable presentation of, and disposal of, said forceps and or bullet and or surgical needle and suture, and or other surgical sharp implements such as a scalpel blade, in such a manner to facilitate the surgical and suturing process, and to eliminate further the possibility of percutaneous injuries. The system will comprise some or all aspects of the items described herein, and related packaging to facilitate the effective use of the system.

It is also apparent that the forceps according to the present invention is not only useful in “open surgery,” its advantages may be exploited in endoscopic surgical procedures or in combination with other endoscopic tools wherein a single arm with a bullet attached at a distal end is employed. The bullet simultaneously supports the tissue to be pierced with a surgical needle and is capable of receiving and affixing the needle thereafter so that the needle can be manipulated with the instrument.

Further advantages of the invention will be appreciated with reference to the appended claims.

### BRIEF DESCRIPTION OF THE DRAWINGS

The invention will now be further elucidated by way of exemplary embodiments that form no limitation to the appended claims, and with reference to the following drawings.

In the drawings:

FIG. 1 shows a perspective view of a surgical instrument according to the invention;

FIG. 2 shows a side elevation of the surgical forceps shown in FIG. 1;

FIG. 3 shows the surgical forceps depicted in FIG. 1 wherein a holder with a bullet is detached from the forceps;

FIGS. 4-7 show several successive stages of using the surgical forceps according to the invention while suturing tissue;

FIG. 8 shows a diagram illustrating the wire mesh embodiment of the bullet;

FIG. 9 shows a diagram illustrating the form lock embodiment of the bullet;

FIG. 10 shows a diagram illustrating the saloon doors embodiment of the bullet;

FIG. 11 shows a perspective view of the preferred embodiment of the surgical forceps with a double hinge mechanism in the closed position;

FIG. 12 shows a perspective view of the preferred embodiment of the surgical forceps with a double hinge mechanism in the open position;

FIG. 13 shows perspective views of the preferred embodiment of the surgical forceps with a double hinge in both open and closed positions;

FIG. 14: shows a diagram illustrating the spring enforced sliding arm embodiment of the surgical forceps;

FIG. 15 shows a diagram illustrating the flipping bullet embodiment of the surgical forceps;

FIG. 16 shows a diagram illustrating the accentric axis embodiment of the surgical forceps;

FIG. 17 shows a diagram illustrating the double spring embodiment of the surgical forceps;

FIG. 18 shows a schematic view of a mini-forceps embodiment of the surgical forceps in open and closed positions;

FIG. 19 shows a perspective view illustrating the mini-forceps embodiment; and

FIG. 20 shows a schematic view of the attachment of the mini-forceps to a surgical instrument.

FIGS. 21-23 show several embodiments of endoscopic surgical forceps for minimally invasive surgery.

### DETAILED DESCRIPTION OF THE INVENTION

The invention is directed to a surgical instrument (suturing forceps), with which needle perforation accidents can be avoided, tissue damage during suturing can be reduced and which allows suturing to be performed more easily.

The following description is made by way of example and is not intended to limit the invention as set out in the appended claims.

The forceps of the present invention comprises at least a first and second arm that are connected at one end and which may be biased, for example, by a spring means, in an open position and which defines a space between them which can be reduced or increased. The instrument also comprises a needle receiving and affixing bullet that is preferably positioned at the distal end, at an inside and/or lower side of the end of an arm. The term “open” in the context of the present invention refers to the position wherein the distal ends of the two arms are apart. The term “closed” refers to the position wherein the distal ends of the two arms are in close proximity or touching.

The term “form lock” refers to the properties of a bullet embodiment whereby the deformation of a bullet that is pierced by a surgical needle results in a pressure exerted on the surgical needle from the resistance of the bullet to return to its original form thereby affixing (locking) the surgical needle to the bullet.

When suturing tissue using a suture and surgical needle, the forceps of the present invention makes it possible to control the suturing process in such a manner that immediately after the point of the surgical needle has pierced the tissue, it is able to pass into the bullet where it is retained until removal by the surgeon, preferably with the use of a surgical needle holder. This allows the surgical needle to be manipulated safely during suturing without touching the needle with the hands, thereby reducing the possibility of needle perforation accidents.

The bullet also provides support for the portion of tissue being sutured by using the forceps of the present invention to effectively avoid tissue damage because the bullet provides a counter pressure against the pressure of the needle supporting the tissue to be sutured thereby minimizing tissue stretch. Furthermore, the suturing operation can be continued using the forceps to manipulate the needle, without the necessity of either manually touching the needle or using a needle holding instrument.

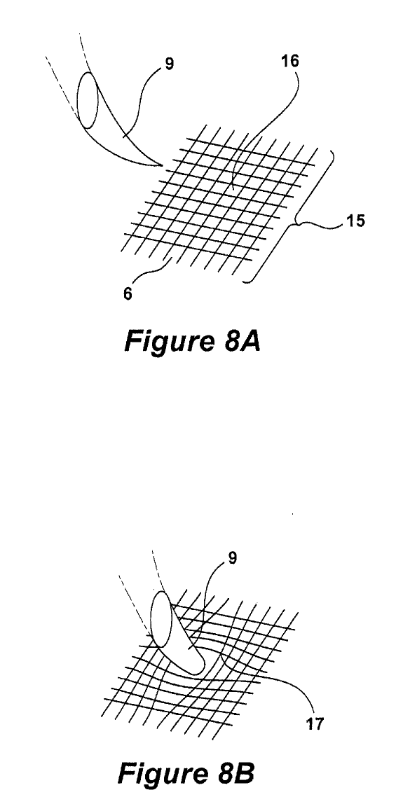

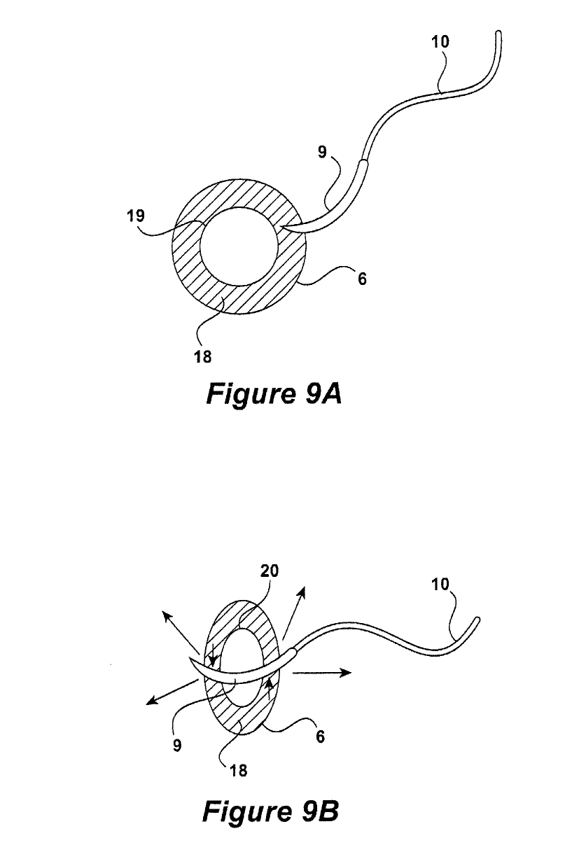

The bullet may be comprised of any material or designed in any way such that it is capable of receiving and removably affixing or retaining a surgical needle. For example, the material may be pierceable such as synthetic rubber, soft plastic, or wire mesh. An example of a bullet comprised of wire mesh is illustrated in FIG. 8. The bullet may also be comprised of a hollow synthetic material such that when penetrated, the pressure of the needle penetrating the first wall of the bullet deforms the bullet, and this pressure, together with the pressure created by the penetration and subsequent deformation of the second wall creates significant pressure on the needle creating a “form lock,” enhancing the bullet's grip on the needle. An example of the form lock embodiment of the bullet is illustrated in FIG. 9. Alternatively the hollow space may be filled with a substance such as a gel that may contribute to “locking” a surgical needle. The bullet may also be comprised of a material with magnetic properties suitable for receiving and retaining a surgical needle or any other receiving and retaining means as with an adhesive.

Other means such as inducible gripping mechanisms may also be used to receive and removably affix a needle. For example, the bullet may be designed with an opening that allows a surgical needle to be inserted frictionlessly into the bullet such that when the distal ends of the arms are apart, a plunger mechanism which is dependent on the distance between the distal ends of the arms is activated and pushes on the part of the needle that is through the opening. The pushing force of the plunger acts as a guillotine and results in a grip on the needle thereby affixing the needle to the bullet. Conversely, when the distal ends of the arms are in close proximity, the plunger mechanism responds by retracting the plunger, removing the pressure on the needle, and the needle is released. Alternatively, the plunger mechanism may be independent of the distance between the distal ends of the forceps and may be activated manually by the surgeon. The bullet may also be comprised of a substance, for example, a soft gel, that receives a surgical needle which by applying or inducing a change in temperature at the site of the bullet, for example by using a laser, causes it to harden thereby fixing the surgical needle that can be released by again applying or inducing a change in temperature at the site of the bullet for example by extinguishing the laser light. The bullet of the above-mentioned embodiments may be comprised of a biodegradable material in the event that if any part of the bullet falls into the wound, no additional harm would be caused to the patient, as the bullet would harmlessly dissolve in the body.

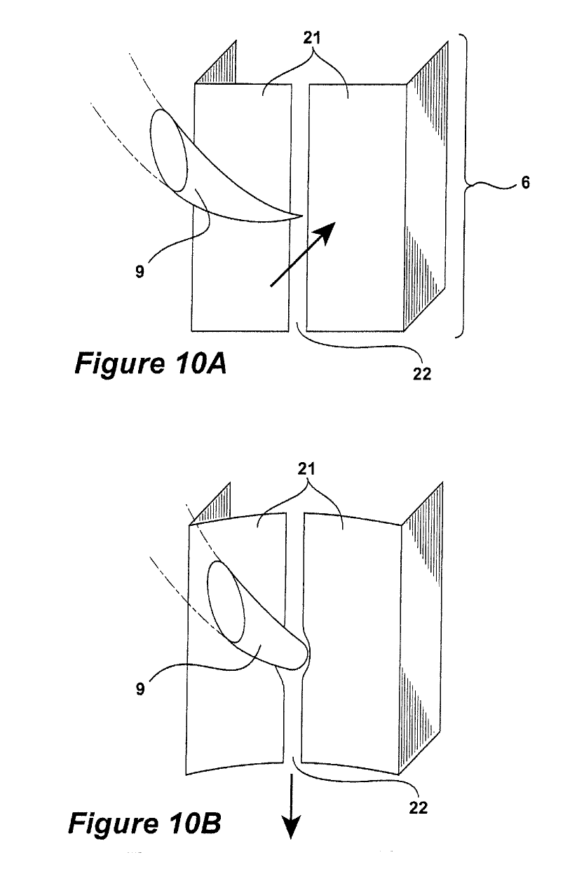

The bullet may also be designed to have a narrow slit into which a surgical needle is guided such that when the forceps are manipulated to pull on the needle, flaps of the slit close thereby affixing the needle to the bullet. The needle is released by pulling or pushing it in the direction of the flaps thereby opening the flaps. Such an embodiment may be referred to as a “saloon door” mechanism. An example of this embodiment is illustrated in FIG. 10.

In a further aspect of the invention, the bullet may be “loaded” with an electric or other charge or with receptors to guide the needle to the bullet such that the needle is controlled and gripped more easily.

One form of inducible gripping means may be actuated by pressure on the bullet due to the normal forces that are on the bullet when used to suture. When the forceps is closed, e.g. on holding tissue, the bullet is deformed by the applied closing pressure and thereby transformed to a more open configuration that allows the needle to be inserted or extracted more easily. When the forceps is opened, the bullet returns to its natural form and in that form the resistance on the needle is increased, making it more difficult to pull the needle out or pierce it in, however facilitating manipulation of the needle by manipulation of the forceps. This considerably aids the normal actions during suture such as when the needle is pulled through tissue.

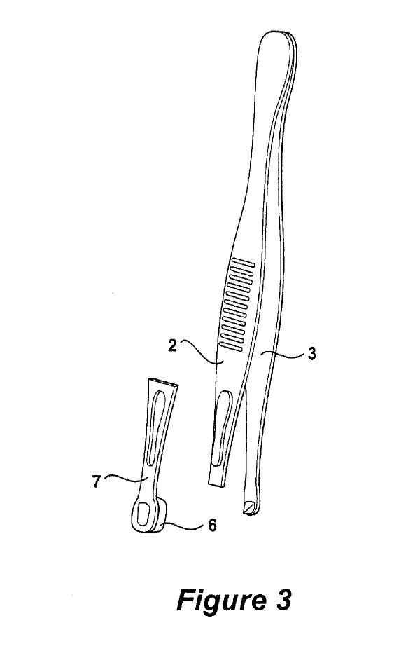

In a further aspect of the invention the bullet is provided on a holder that is detachably placed on at least one arm as exemplified herein. The holder with the bullet may thus be a disposable component that can be supplied sterile, while the forceps upon which the holder is placed may be retained and sterilized from case to case. The holder with the bullet is designed to be removably placed on forceps of the kind illustrated in FIG. 3.

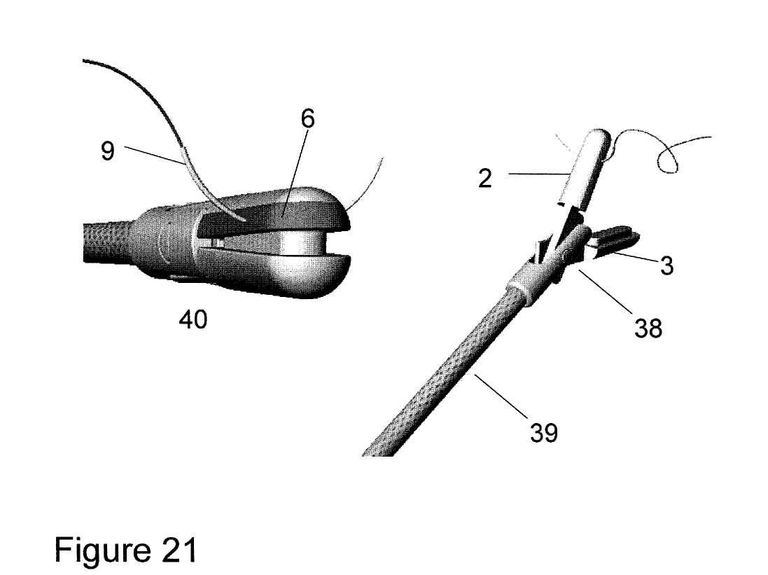

In certain embodiments, the present invention provides an endoscopic forceps for minimally invasive or robotic surgery. The purpose of this forceps is to hold a surgical needle firmly while the user knots the suture and while the suture needle is transferred during endoscopic or robotic or minimally invasive, surgery. In one such embodiment, the forceps comprises a proximal control device for manually operating the device coupled to a distal operative device, the operative device and control device being separated by a shaft and the operative device comprising a first forceps arm (or jaw) and a second forceps arm (or jaw) hingeably attached at the proximal end to a distal portion of the device and defining a space between them which can be increased or reduced by operation of the control device. The forceps further comprises a bullet at the distal end of at least at the first jaw and/or second jaw, at an inside and/or lower side of the distal end of a jaw to receive and affix a surgical needle while gripping the tissue. The location of the bullet on a first and/or second forceps jaw, such that the bullet is capable of receiving and affixing the needle while the tissue gripped, allows the surgical needle to be manipulated more easily during suturing, thereby reducing the possibility of needle drop accidents and tissue damage accidents. FIGS. 21-23 illustrate various embodiments of the forceps for minimally invasive surgery.

In preferred embodiments, the bullet forms part of the tissue-gripping surface of the endoscopic forceps. In these embodiments, the bullet is positioned such that the bullet simultaneously supports the tissue to be pierced with a surgical needle and is capable of receiving and affixing the needle thereafter so that the needle can be manipulated with the instrument. In these embodiments, the bullet is preferably positioned at an inside of the first and/or second forceps jaw, but may be placed at any position on a jaw that allows the bullet to simultaneously grip the tissue and receive and affix a surgical needle. The bullet may also be integrated into a first and/or second forceps jaw.

In other related embodiments, the bullet does not form part of the tissue gripping surface of the endoscopic forceps; however, the bullet is positioned such that the bullet is capable of receiving and affixing the needle while the forceps jaws grip the tissue. In these embodiments, the needle-affixing material may be placed at an outside of the first and/or second forceps jaw. The bullet may also be integrated into a first and/or second forceps jaw.

In using the present invention there is a moment in suturing in which the surgeon pulls the needle (that is already fixed in the bullet) through the tissue. In order to do so without harming the tissue, the surgeon guides the forceps following the curvature of the needle. By doing so, the arm opposite to the arm with the bullet to which the needle is affixed can touch or even get stuck in the tissue. Providing a means for moving the opposite arm away from the surgical field when the distal ends of the two arms are apart alleviates this problem.

To this end, a preferred embodiment of the invention is comprised of the two arms of the forceps being of unequal length with a hinge mechanism, which is comprised of a hinge and a lever, attached to the shorter arm that is opposite the bullet.

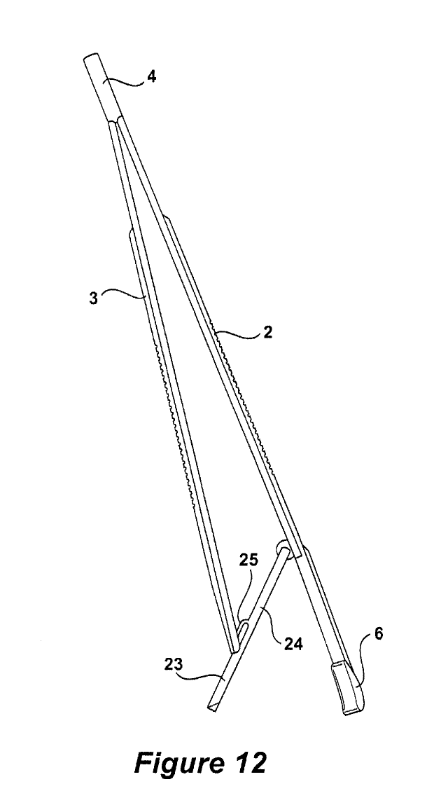

When the forceps are in a closed position, the distal end of lever extends to contact the distal end of the arm to which the bullet is attached. When in the open position, the lever retracts and is no longer in the way of making a circular motion with the forceps to pull the needle through the tissue along the curvature of the needle. Such an embodiment may be referred to as “double-hinge.” An example of this embodiment is illustrated in FIG. 12.

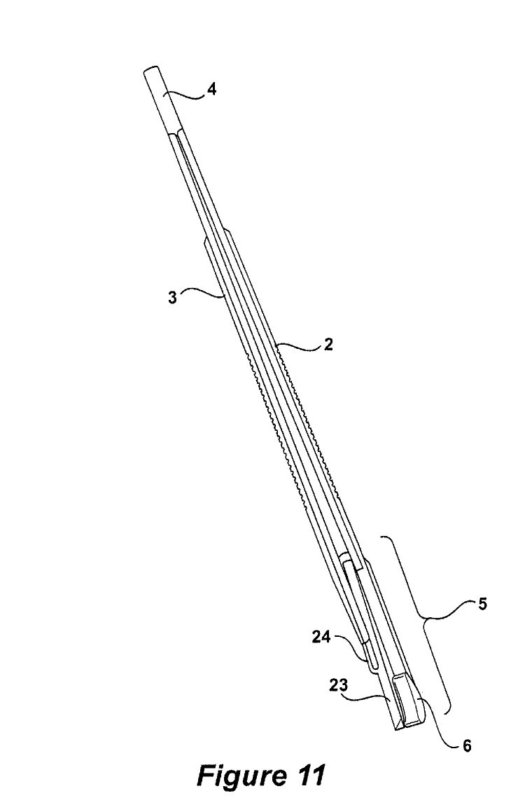

For this embodiment, the hinge mechanism is comprised of a hinge that is medially fixed to a lever. The hinge is also fixed at the distal end of the shorter arm opposite the bullet such that the distal end of the lever acts as an extension of the shorter arm. The proximal end of the lever is slidably disposed along the inside of the longer arm. Examples of this embodiment are illustrated in FIGS. 11, 12 and 13.

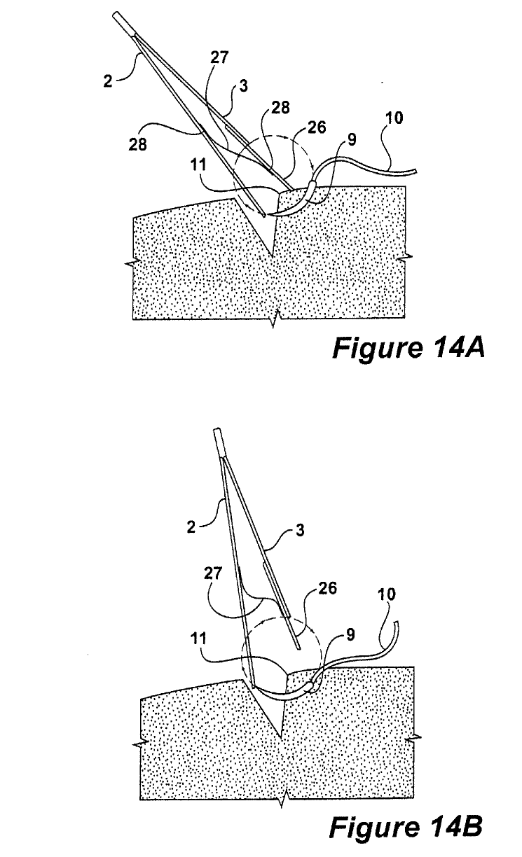

In an alternate embodiment of the invention, the hinge mechanism is in the form of a spring medially fixed to a sliding lever. The spring opens the forceps and by motion of the sliding lever makes the distal end of the arm that is to be moved away (i.e. the arm opposite the arm with the bullet) slide in the proximal direction when the forceps are open. When the forceps are closed, the sliding lever slides towards the distal end of the forceps to enable sufficient grip of the tissue. Such an embodiment may be referred to as “spring enforced sliding arm.” An example of this embodiment is illustrated in FIG. 14.

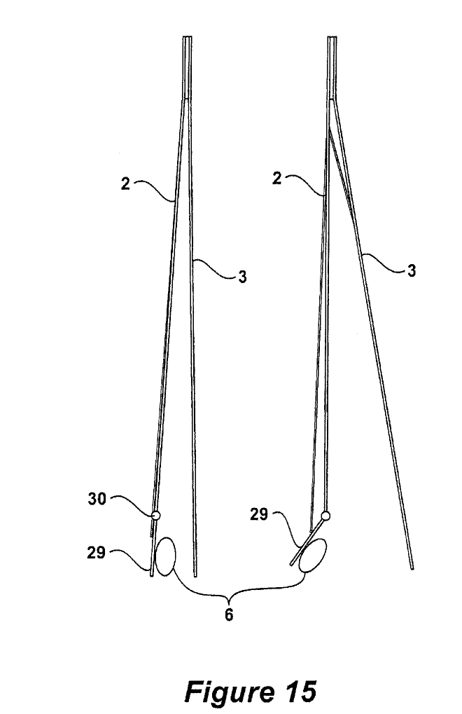

In an alternate embodiment of the invention, the hinge mechanism is fixed to the distal end of the shorter arm of the forceps. Furthermore, the bullet is placed at the distal end of the hinge mechanism. Spring-forced movement of the hinge flips it away from the longer arm of the forceps and allows the surgeon to pull the surgical needle through, along the natural needle path, without damaging the tissue. Such an embodiment may be referred to as “flipping bullet.” An example of this embodiment is illustrated in FIG. 15.

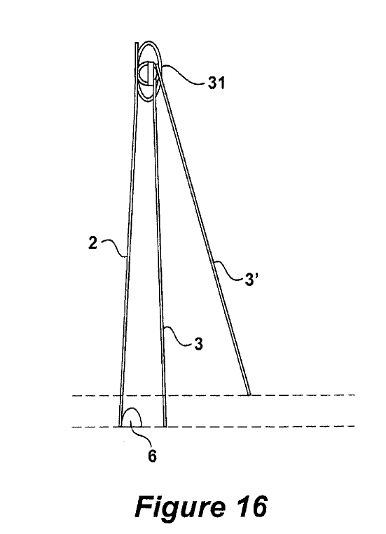

In an additional embodiment of the invention, an elliptical hinge mechanism at the proximal end of the forceps may be used in which the point of rotation in the proximal end makes a translating movement at the same time as the rotating movement takes place. This design “shortens” the arm that is opposite the bullet when the forceps are opened. Such an embodiment may be referred to as “accentric axis.” An example of this embodiment is illustrated in FIG. 16.

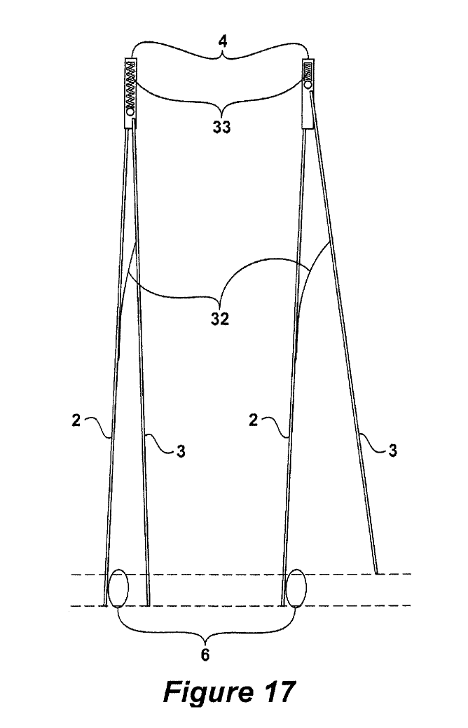

In an additional embodiment of the invention, the hinge mechanism is comprised of a double spring. A first spring holds the two arms apart in the open position in which the arm opposite that which holds the bullet is shorter. When the first spring is engaged and the distal ends of the two arms are brought together, a second spring located at the proximal end of the instrument and fixed to the shorter arm is also engaged and extends the shorter arm such that the distal ends of the two arms meet when the forceps are in the closed position. Such an embodiment may be referred to as “double spring.” An example of this embodiment is illustrated in FIG. 17. Other means by which to accomplish shortening of the arm of the forceps will be readily apparent to one of ordinary skill in the art.

Identical reference numerals used in the figures refer to similar parts.

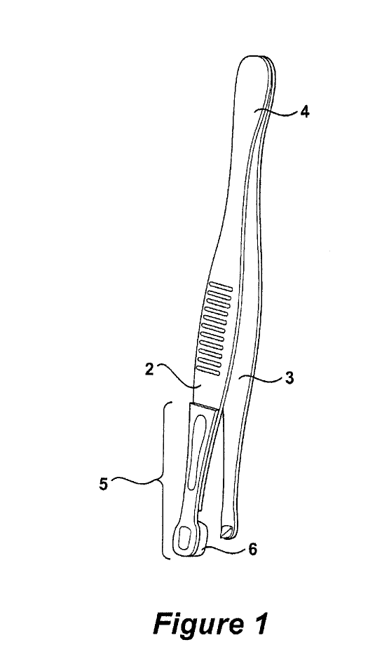

Referring first to FIG. 1, where reference numeral 1 indicates the surgical forceps according to the invention.

These surgical forceps 1 are suitable to be used for suturing tissue and comprise a first forceps arm 2 and a second forceps arm 3, spring-connected at a proximal end 4, i.e. the end which during the manipulation of the forceps 1 lies in the hand and which arms define a space between them which an be reduced and increased.

At a distal end 5, the first forceps arm 2 and the second forceps arm 3 can be moved toward each other.

FIG. 1 further shows that the first forceps arm 2 is provided with a bullet 6. This bullet 6, in its preferred embodiment, is comprised of a needle receiving and retaining material such an elastomeric material which is suitable to be pierced with a surgical needle and which removably retains the needle until removed by the surgeon, as will be further explained below.

The bullet may also be provided on the second arm 3 or, as the case may be, only on the second arm 3. Within the framework of the invention, however, at least one of the forceps arms 2, 3 must be provided with a bullet 6.

As FIG. 1 shows, the bullet 6 is positioned close to or at the distal end 5, at an inside or lower side of the end of the first forceps arm 2.

The bullet 6 in one of its embodiments is preferably designed to be able to receive and affix a surgical needle pierced therethrough. A material to be used as the bullet 6 is suitably a synthetic material, for example synthetic rubber or other elastomeric material. Advantageously, the bullet 6 together with the end of the arm upon which it is placed define a space between the first arm 2 and the second arm 3 which can be reduced or increased. The fabrication of this is well known to the person skilled in the art and requires no further elucidation.

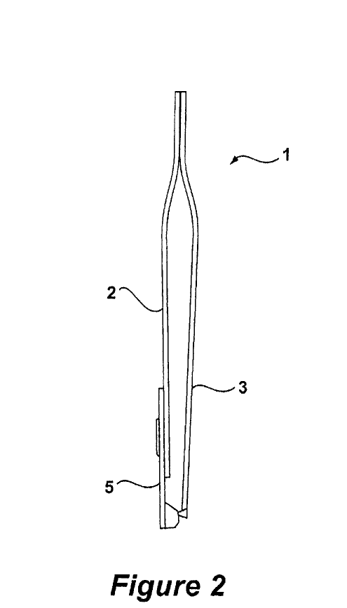

FIG. 2 shows a side elevation of the surgical forceps 1 according to the invention wherein the first forceps arm 2 and the second forceps arm 3 are moved toward each other.

FIG. 3 shows that the bullet 6 is provided on a holder 7 that is detachable from but, as in the illustrated case, can also be detachably placed on the first forceps arm 2. Any means of attachment and detachment may be used including that illustrated in FIG. 3, screw on and off attachment means, clip on, luer-lock and others.

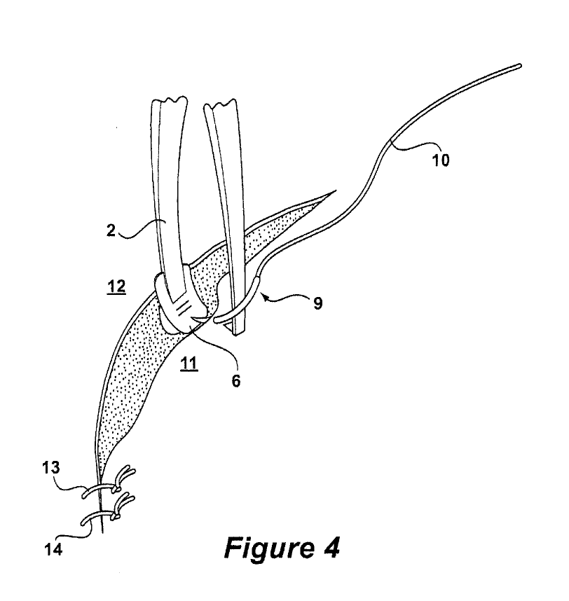

The use of the surgical forceps 1 according to the invention may conveniently be explained by way of a series of successive steps illustrated in the FIGS. 4-7, showing the use of the surgical forceps 1 according to the invention for suturing tissue.

FIG. 4 shows a first step, wherein by means of a needle-holding tool (not shown) a surgical needle 9, attached to which is a suture 10, pierces a first tissue portion 11 in order to join this first tissue portion 11 with a second tissue portion 12.

Reference numerals 13 and 14 indicate two sutures made previously through the first and second tissue portions 11 and 12.

FIG. 4 shows clearly that the first forceps arm 2, which at the inside distal end is provided with a bullet 6, serves to support the first tissue portion 11 through which the suture 10 is passed. In this way the surgical forceps 1 according to the invention are able to effectively support the first tissue portion 11 so as to avoid damage to this first tissue portion 11, while simultaneously a point of the surgical needle 9 is able to pass into the bullet 6 in order to receive and affix the surgical needle 9 therein.

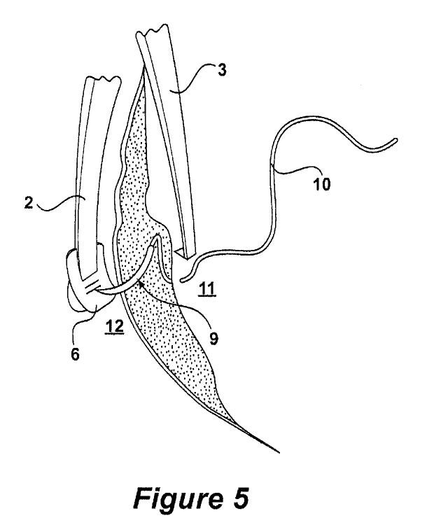

FIG. 5 subsequently shows that the surgical needle 9 can be passed further through the first tissue portion 11 by employing the surgical forceps 1 in accordance with the invention.

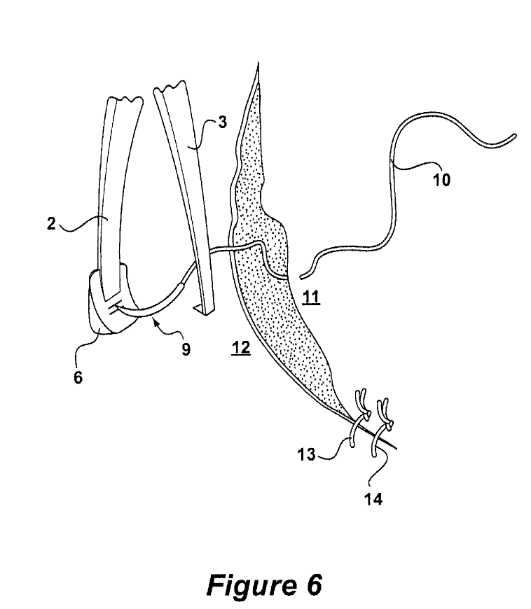

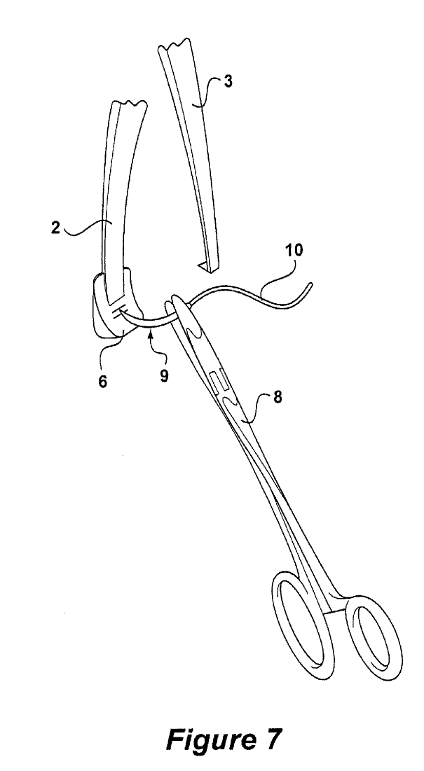

FIG. 6 subsequently shows that the surgical needle 9, with the suture 10 attached thereto, is in an advanced stage of its passage through the first tissue portion 11 and as FIG. 7 further shows, that the surgical needle 9 thus becomes available again for manipulation by using a needle-holding tool 8.

FIGS. 8-10 show embodiments of various mechanisms by which the bullet 6 may receive and affix a surgical needle 9, FIG. 8 shows a wire mesh embodiment of the bullet 6 wherein a tightly woven mesh with wires 15 that can slide in relation to each other, with at some intervals no sliding knots between wires. The surgical needle 9 is inserted in one of the pores 16 resulting in displacement of the wires until a non-moving corner 17 is encountered. The interval of the non-moving corner 17 assists in the grip on the needle 9.

FIG. 9 shows a “form lock” embodiment wherein a bullet 6 is comprised of a synthetic rubber material with a hollow core. The bullet may be of any shape. The act of inserting a surgical needle 9 through the first layer 18 and subsequently through an open space 19 and then through a second layer 20 of the bullet 6 causes a deformation of the bullet 6. The physical dynamics of the bullet 6 trying to return to its neutral shape, due to its material memory, causes increased pressure to be borne on the needle 9, thereby increasing the grip the bullet 6 has on the needle 9.

FIG. 10 shows a “saloon doors” embodiment of the bullet 6 wherein the surgical needle 9 is stuck between two pieces of material or flaps 21 that have a very narrow slit 22 in between. The needle 9 is guided to go between the two flaps 21, resulting in “opening the saloon doors.” When the forceps are manipulated to pull the surgical needle 9 out of the tissue, the flaps 21 close, resulting in a grip on the surgical needle 9 because of the additional space the needle 9 occupies between the flaps 21. The greater the pulling force applied, the stronger the grip on the needle 9 because of the friction between the needle 9 on the flaps 21 forces the flaps to close further. After the needle 9 is pulled through the tissue, the needle 9 is released by pulling or pushing it in the direction the flaps 21 open.

The remaining figures address the potential problem encountered in suturing in which the surgeon wants to pull the needle (that is affixed to the bullet) through the tissue. In order to do so without harming the tissue, the surgeon will want to guide the forceps following the curvature of the needle. By doing so, the arm opposite the arm with the flexible material can touch or even get stuck in the tissue.

FIGS. 11-13 illustrate the surgical forceps with a double hinge mechanism. In FIG. 11, the instrument is comprised of a longer first arm 2 and a shorter second arm 3 with a hinge mechanism that includes a lever 23 and a hinge 24 fixed to the distal end 5 of the second arm 3. When combined with the lever 23, the distal end of lever 23 contacts the distal end of the first arm 2 when the instrument is in the closed position.

FIG. 12 shows the surgical forceps 1 with the hinge mechanism comprised of a lever 23 and a hinge 24 in the open position. A spring 25 may be used to enforce the open position when the forceps are not engaged.

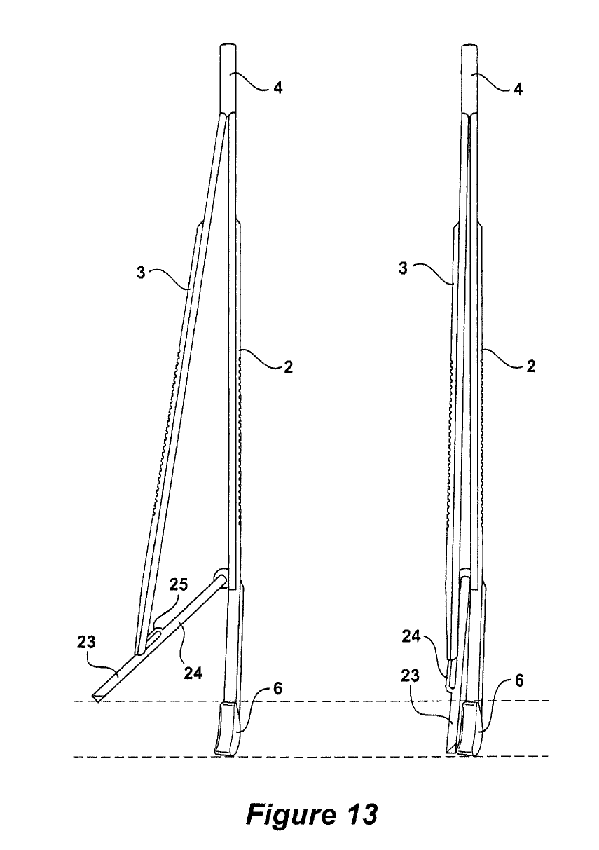

FIG. 13 shows the surgical forceps 1 in both the open and closed positions. Note the difference in length of the second arm 3 combined with the lever 23 along the axis of the forceps when in open and closed positions. In the open position, the lever 23 is out of the way when making a circular motion with the forceps to pull a needle (not shown) through tissue along the curvature of the needle thereby avoiding unwanted contact with and/or damage to the tissue.

FIG. 14 shows the spring enforced sliding arm embodiment of the surgical forceps 1 wherein the second arm 3 is shorter than the first arm 2. The second arm 3 has attached to it at the distal end a sliding lever 26. A spring 27 is fixed to a medial region 28 of the first arm 2 and the sliding lever 26 such that it makes the distal end of the sliding lever 26 slide in the proximal direction when the forceps are in the open position. When the forceps are closed, the sliding lever 26 slides towards the distal end of the instrument to enable sufficient grip of the first tissue portion 11 to be sutured.

FIG. 15 illustrates the flipping bullet embodiment of the invention wherein a short arm 29 to which a bullet 6 is attached is fixed at the distal end of the first arm 2 by a hinge 30. The first arm 2 is shorter than the second arm 3, but in the closed position, the distal end of the short arm 29 to which the bullet 6 is attached touches the distal end of the second arm 3. Spring-forced movement of the hinge 30 that the surgeon can manipulate flips the short arm 29 away from the longer second arm 3 of the forceps and allows the surgeon to pull the surgical needle (not shown) through, along the natural needle path, without damaging the tissue.

FIG. 16 illustrates the accentric axis embodiment of the invention wherein an elliptical hinge mechanism 31 in which the point of rotation in the proximal end of the forceps makes a translating movement at the same time as the rotating movement takes place. Under this design, when the forceps are in the closed position, the distal end of the second arm 3 extends such that it meets the distal end of the first arm 2. In the open position, the elliptical hinge mechanism 31 shortens the second arm 3′, thereby avoiding damage to the tissue.

FIG. 17 illustrates the double spring embodiment of the invention wherein the second arm 3 that is opposite the bullet 6 is retracted and extended by a first spring 32 that holds the first arm 2 and second arm 3 apart in the open position in which the second arm 3 is shorter than the first arm 2. When the first spring 32 is engaged and the distal ends of the two arms are brought together, a second spring 33 located at the proximal end 4 of the forceps and fixed to the shorter second arm 3 is also engaged and extends the shorter second arm 3 such that the distal ends of the first arm 2 and second arm 3 meet when the instrument is in the closed position.

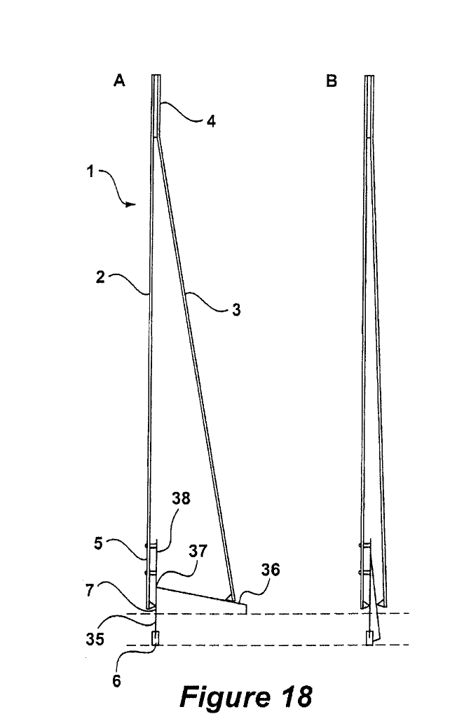

FIGS. 18 A, B 19 and 20 illustrate a mini-forceps embodiment of the present invention which operates in a similar manner to the double-hinge mechanism of FIGS. 11-13. It has the additional advantage that the holder 7 on which the bullet 6 is mounted may be formed as a disposable item for connection to a standard medical forceps or other suitable holder/actuator.

FIG. 18A shows a schematic view of the mini forceps embodiment in the open position. According to FIG. 18A, a standard surgical forceps 1 comprises a first arm 2 and a second arm 3, spring-connected together at a proximal end 4. A bullet holder 7 carrying bullet 6 is releasably connected to the distal end 5 of the first arm 2. The holder 7 is formed as a mini-forceps and comprises a first member 35 and second member 36 joined together at their proximal ends by a hinge 37. The bullet 6 is provided at the distal tip of the first member 35. A connecting member 38 extends proximally from the first member 35 for connection to the forceps 1.

FIG. 18B shows a schematic view of the mini forceps embodiment in closed position. As can be seen from the figure, the limited length of the first and second members 35, 36 relative to the arms 2, 3 ensures that the angular movement of the mini-forceps 7 is considerably greater than that of the forceps 1 on movement between closed and open positions. This increased angular movement ensures that in use, the second member 36 is distanced from the path of movement of the surgical needle 9.

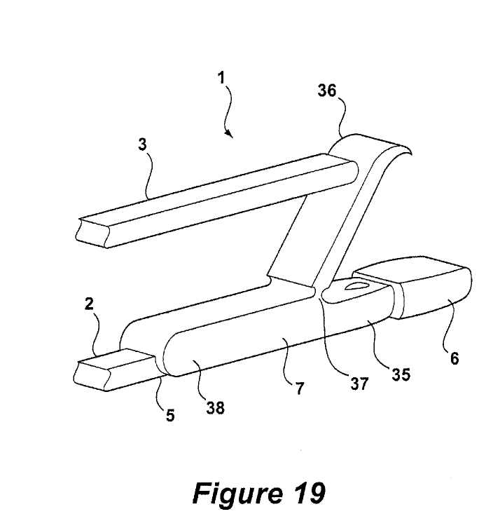

FIG. 19 shows a perspective view of the holder 7 which is formed of a suitable medical grade plastics material. The hinge 37 is formed as a living hinge having a resilient bias to an open position. It is evident that alternative materials could be used for forming the holder and hinge and that its construction as a disposable device is merely optional. Furthermore, various connecting mechanisms may be envisaged for attaching the connecting member 38 to the forceps 1.

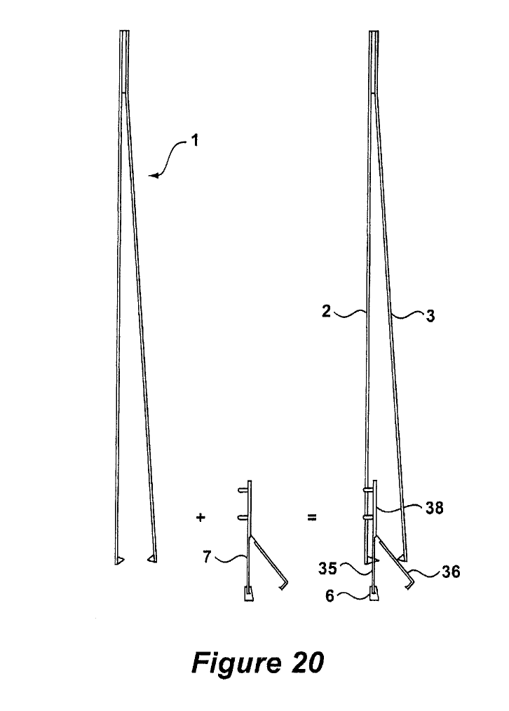

FIG. 20 shows a schematic view of the mini forceps embodiment illustrating how the holder (mini forceps) 7 may be connected to e.g. a standard medical forceps 1 to form a combined instrument.

FIG. 21 depicts an embodiment of an endoscopic forceps 1 for minimally invasive surgery. The forceps comprises a proximal control device (not shown) for manually operating the forceps coupled to a distal operative device 38, the operative device and control device being separated by a shaft 39 and the operative device comprising a first arm (or jaw) 2 and a second forceps arm (or jaw) 3 hingeably attached at the proximal end to a distal portion of the device and defining a space between them which can be increased or reduced by operation of the control device. The inner surface of the first jaw 2 comprises a bullet 6 comprising a soft needle-affixing material (e.g. soft plastic or synthetic rubber) through which a needle 9 may be guided. The inner surface of the second jaw 3 comprises a plunger 40 which exerts a force on the needle 9 when the forceps 1 are in a closed position, thus clamping the needle.

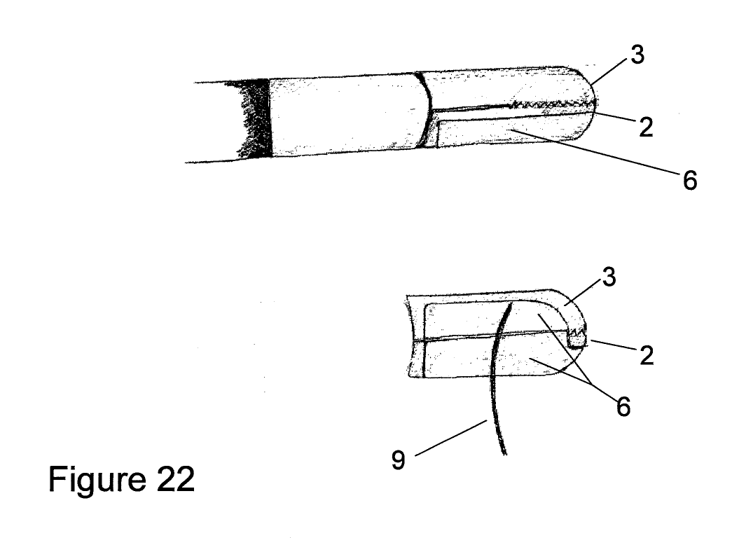

FIG. 22 illustrates related embodiments of an endoscopic forceps 1 for minimally invasive surgery. The top panel illustrates an embodiment in which the bullet 6 comprising a soft needle-affixing material is placed on an outside surface of the first forceps jaw 2. The bottom panel illustrates an embodiment in which the first forceps jaw 2 comprises an O-shaped ring into which a bullet 6 comprising a soft-needle-affixing material is placed and the second forceps jaw 3 comprises a bullet 6 comprising needle-affixing material on an inner surface 41 of the jaw. In this embodiment, a needle 9 may be inserted through the needle-affixing material in the O-shaped ring of the first jaw 2 and continue through the needle-affixing material of the second jaw 3 until it reaches the inner surface of the second jaw 3. The second jaw 3 is made of a needle resistant material to prevent the needle from exiting the bullet.

In this embodiment, the bullet may be used to fully cover the needle tip during all stages of endoscopic surgery including entry of the needle into, and exit of the needle from, the body cavity. Entry of a suture needle into the body cavity during endoscopic surgery requires penetration through a tube (trocar) which contains an air-tight valve at the entry site. If the needle tip is exposed it can cause a leak as it passes through the valve. This embodiment may effectively prevent such leaks by covering the needle tip during entry. This embodiment may also prevent accidental perforation of the bowel or other tissue during the endoscopic surgerical procedure by covering the needle tip.

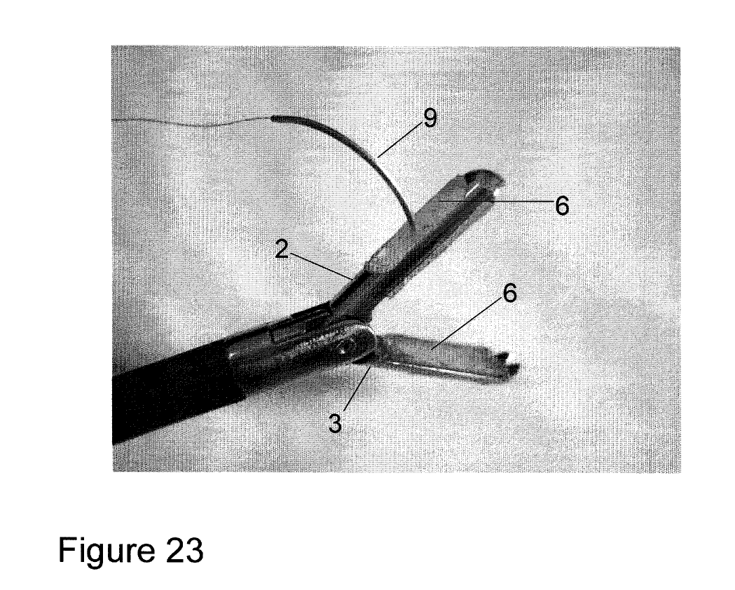

FIG. 23 illustrates a related embodiment of a device for minimally invasive (e.g. endoscopic) surgery. In this embodiment, the first forceps jaw 2 and the second forceps jaw 3 each comprises an O-shaped ring into which a bullet 6 comprising a needle-affixing material is placed.

1. An endoscopic forceps to be used for suturing tissue, comprising a proximal control means for manually operating the forceps coupled to a distal operative means, the operative means and control means being separated by a shaft and the operative means comprising a first forceps jaw and a second forceps jaw hingeably attached at the proximal ends to a distal portion of the device and defining a space between them which can be increased or reduced by operation of the control means, said forceps further comprising a bullet placed at a distal end of said first jaw and/or said second jaw, at an inside and/or lower side of the end of said jaw, to receive and affix a surgical needle while gripping the tissue.

2. The forceps of claim 1, wherein said bullet is placed at a distal end of said first jaw and/or said second jaw, at an inside of the end of said jaw.

3. The forceps of claim 1, wherein said bullet is placed at a distal end of said first jaw and/or said second jaw, at a lower side of the end of said jaw.

4. The forceps of claim 3, wherein said bullet is placed at an outside of the end of said jaw.

5. The forceps of claim 1, wherein said bullet is suitable for being pierced with said surgical needle.

6. The forceps of claim 5, wherein said bullet is comprised of elastomeric material.

7. The forceps of claim 6, wherein said elastomeric material is synthetic rubber.

8. The forceps of claim 1, wherein said bullet has a hollow core surrounded by a pierceable layer for affixing said surgical needle by a form lock, whereby the pierced material exerts a force on the inserted needle as a result of its deformation.

9. The forceps of claim 1, wherein said bullet has a core comprised of a filler material said filler selected from gels, foams, beads or liquids surrounded by a pierceable layer for affixing said surgical needle by a form lock, whereby the pierced material exerts a force on the inserted needle as a result of its deformation.

10. The forceps of claim 1, wherein said bullet comprises wire mesh.

11. The forceps of claim 1, wherein said bullet comprises a magnetic material.

12. The forceps of claim 1, wherein said bullet comprises an adhesive material.

13. The forceps of claim 1, wherein said bullet comprises a clamp.

14. The forceps according to claim 1, wherein said bullet comprises an inducible gripping mechanism comprising a plunger mechanism wherein said plunger mechanism is activated as said distal ends of said first jaw and said second jaw move apart toward an open position and where said distal ends of said jaws are in close proximity, said plunger mechanism is retracted thereby releasing said needle.

15. The forceps of claim 14, wherein said plunger mechanism is manually activated independent of said open or said closed position of said forceps.

16. The forceps of claim 1 wherein said bullet is comprised of a hollow structure comprised of at least two flaps separated by a narrow slit into which said surgical needle may be guided said flaps being suitable for affixing said surgical needle to said bullet and where said surgical needle is released from said bullet by pulling said surgical needle through.

17. The forceps of claim 1 wherein said bullet is comprised of a material that responds to changes in chemical or physical conditions such as temperature whereby said bullet affixes and releases said surgical needle upon a change in chemical or physical conditions at site of said bullet.

18. The forceps of claim 2 wherein said forceps comprises a bullet placed at a distal end of said first jaw at an inside of the end of said first jaw and comprises a plunger placed at an inside of the end of said second jaw, wherein said plunger exerts a force on said surgical needle affixed to said bullet when said forceps are in a closed position.

19. An endoscopic forceps to be used for suturing tissue, comprising a proximal control means for manually operating the forceps coupled to a distal operative means, the operative means and control means being separated by a shaft and the operative means comprising a first forceps jaw and a second forceps jaw hingeably attached at the proximal ends to a distal portion of the device and defining a space between them which can be increased or reduced by operation of the control means, said first jaw and/or said second jaw having an O-shaped ring at a distal end of said jaw comprising a bullet to receive and affix a surgical needle while gripping the tissue.

20. The forceps of claim 19, said first jaw and said second jaw having an O-shaped ring at a distal end of said jaw comprising a bullet to receive and affix a surgical needle while gripping the tissue.

21. The forceps according to claim 1 wherein said bullet is provided on a holder that is removable from said arm.

22. The forceps according to claim 1 wherein said bullet is comprised of a biodegradable material.

23. A method for suturing tissue using the forceps according to claim 1, comprising the steps of securing and supporting a first area of tissue to be sutured with said distal end of said forceps, securing said surgical needle and a suture material attached thereto with a needle holding tool, piercing said first area of tissue to be sutured with said surgical needle using said bullet to support said tissue, passing said surgical needle through said tissue and affixing said surgical needle to said bullet of said forceps while securing said first area of tissue with said distal end of said forceps, releasing said surgical needle form said needle holding tool, releasing said first area of tissue secured by said forceps, guiding the surgical needle with said forceps following the curvature of said surgical needle, and removing said surgical needle from said bullet of said forceps by securing said surgical needle with said needle holding tool.

24. The method of claim 23, wherein said method is repeated on a second area of tissue to be sutured to said first area of a lesion whereupon said suture passing through said first area of tissue and second area of tissue is tied in a knot.

25. The method of claim 23, wherein prior to said bullet receiving and affixing said surgical needle, said first and second areas of tissue to be sutured are pierced by said surgical needle in one movement.

26. The method of claim 23, wherein said suture is knotted by a force applied by said needle holding tool pulling on an end of said suture and second pulling force applied by said forceps to which said surgical needle is affixed.

27. The method of claim 23, wherein said method is performed robotically.
